(19) United States
(12) Patent Application Publication
(10) Pub. No.: US 2022/0005575 A1
(43) Pub. Date: Jan. 6, 2022

(54) INFORMATION PROCESSING DEVICE, INFORMATION PROCESSING METHOD, AND NON-TRANSITORY RECORDING MEDIUM

(71) Applicant: CASIO COMPUTER CO., LTD., Tokyo (JP)

(72) Inventor: Masashi UEDA, Tokyo (JP)

(73) Assignee: CASIO COMPUTER CO., LTD., Tokyo (JP)

(21) Appl. No.: 17/361,533

(22) Filed: Jun. 29, 2021

(30) Foreign Application Priority Data
Jul. 1, 2020 (JP) 2020-113791

(51) Int. Cl. G16H 20/30; G01P 15/18; A63B 24/00; G16H 40/63; G06N 20/10; G06F 1/16

(52) U.S. Cl. CPC G16H 20/30; G01P 15/18; A63B 24/0062; A63B 2220/836; G06N 20/10; G06F 1/163; A63B 2220/40; G16H 40/63

(57) ABSTRACT

An information processing device includes an acquirer that acquires exercise data related to exercise of a user from a detector that detects the exercise data, and a determiner that, based on data that is included in the exercise data acquired by the acquired and that indicates acceleration in a specific direction, determines the presence or absence of noise caused by a collision.

### CROSS-REFERENCE TO RELATED APPLICATION

This application claims the benefit of Japanese Patent Application No. 2020-113791, filed on Jul. 1, 2020, the entire disclosure of which is incorporated by reference herein.

### FIELD

The present disclosure relates generally to an information processing device, an information processing method, and a non-transitory recording medium.

### BACKGROUND

In the related art, Japanese Unexamined Patent Application Publication No. 2016-34479, for example, describes a device attached to the waist of a user. The device detects, from an acceleration value and with high accuracy, the timings and the like at which a user that is running leaves and contacts the ground.

### SUMMARY

An information processing device according to some embodiments includes:

at least one processor configured to
acquire exercise data related to exercise of a user from a sensor that detects the exercise data, and
determine, based on data that is included in the acquired exercise data and that indicates acceleration in a specific direction, a presence or absence of noise caused by a collision.

### BRIEF DESCRIPTION OF THE DRAWINGS

A more complete understanding of this application can be obtained when the following detailed description is considered in conjunction with the following drawings, in which:

FIG. 1 is a schematic drawing illustrating a state in which an information processing device according to an embodiment is worn by a user;

FIG. 2 is a block diagram illustrating the configuration of the information processing device according to the embodiment;

FIG. 3 is a graph illustrating change over time of Y-axis direction acceleration when running;

FIG. 4 is a scatter diagram based on feature quantities calculated from the Y-axis direction acceleration;

FIG. 5 is a graph illustrating change over time of Z-axis direction acceleration when running;

FIG. 6 is a scatter diagram based on feature quantities calculated from the Z-axis direction acceleration;

FIG. 7 is a flowchart of index computation processing according to the embodiment;

FIG. 8 is a flowchart of index calculation processing according to the embodiment;

FIG. 9 is a flowchart of first determination processing according to the embodiment; and

FIG. 10 is a flowchart of second determination processing according to the embodiment.

### DETAILED DESCRIPTION

An information processing device according to embodiments is described while referencing the drawings. Note that, in the drawings, identical or corresponding components are denoted with the same reference numerals. The information processing device according to the present embodiment is a device that is worn on the waist of a user, acquires data related to exercise of the user (exercise data), and calculates an index related to the exercise of the user (for example, a magnitude of vertical movement of the body, a rotation angle of the waist, and the like) from the acquired exercise data.

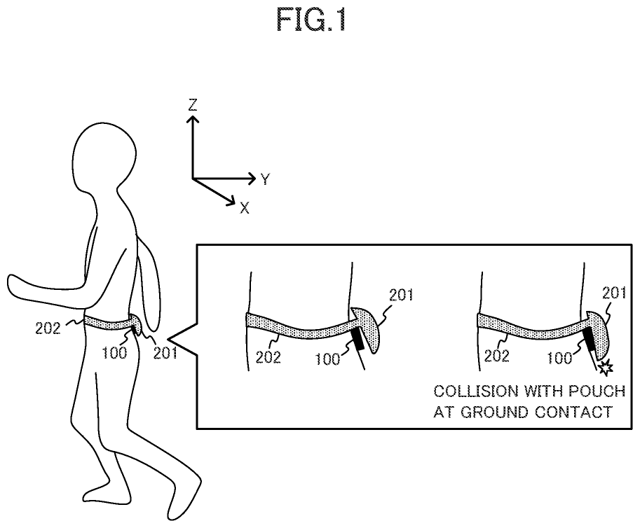

As illustrated in FIG. 1, an information processing device 100 is worn on the waist of the user, detects, by an acceleration sensor, various accelerations in an up-down direction (direction along the Z-axis), a front-back direction (direction along the Y-axis), and a left-right direction (direction along the X-axis) at the wearing portion, and calculates an index related to the exercise of the user on the basis of these acceleration values. Additionally, as illustrated in FIG. 1, the user may wear a pouch 201 on the waist using a pouch belt 202. In such a case, when, for example, the user is performing exercise such as running or jumping rope, the pouch 201 contacts the information processing device 100 the moment a foot of the user makes ground contact, and the acceleration sensor of the information processing device 100 detects a value that is influenced by the collision of the pouch 201. Additionally, the value that is influenced by the collision of the pouch 201 includes noise caused by this influence, which may make it difficult to accurately calculate the index related to the exercise of the user. In other words, in a state in which there are no collisions of the pouch 201 it is possible to accurately calculate the index related to the exercise of the user using the values from the acceleration sensor.

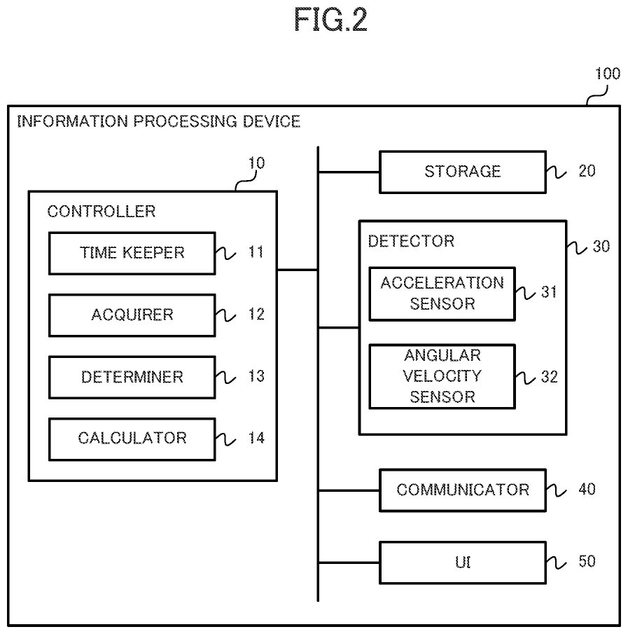

As illustrated in FIG. 2, the information processing device 100 includes a controller 10, a storage 20, a detector 30, a communicator 40, and a UI 50.

In one example, the controller 10 is configured from a processor such as a central processing unit (CPU) or the like. By executing a program stored in the storage 20, the controller 10 functions as a time keeper 11, an acquirer 12, a determiner 13, and a calculator 14, which are described in detail later.

The program that is executed by the controller 10, calculated data, data detected by the detector 30, and the like are stored in the storage 20. The storage 20 can include random access memory (RAM), read-only memory (ROM), flash memory, and the like, but the storage 20 is not limited thereto. Note that the storage 20 may be provided inside the controller 10.

The detector 30 includes sensors for acquiring data needed to calculate the index related to the exercise of the user. Examples of such sensors include an acceleration sensor 31 that detects acceleration, an angular velocity sensor 32 that detects angular velocity, and the like. In one example, the angular velocity sensor 32 is a gyrosensor, but the angular velocity sensor 32 is not limited thereto. It is sufficient that the detector 30 includes at least the acceleration sensor 31, and need not include the angular velocity sensor 32, for example. The detector 30 functions as detection means.

The communicator 40 is implemented as a communication interface that outputs, to an external device (for example, a smartwatch, a smartphone, or the like), data such as the index calculated by the controller 10. The communicator 40 can, for example, include a wireless communication interface or a wired communication interface, but the communicator 40 is not limited thereto. It is possible for the information processing device 100 to send, via the communicator 40, the calculated index related to the exercise of the user to the external device such as a smartwatch.

The UI 50 includes an output device (for example, a display panel) and an input device (for example, a touch panel integrated with the display panel). The UI 50 displays the index related to the exercise of the user calculated by the information processing device 100, receives operations from the user, and the like. Note that the UI 50 is not limited to a touch panel and, for example, may include a press button switch as the input device and a speaker and/or a light emitting diode (LED) as the output device. The UI 50 functions as notification means.

The time keeper 11 of the controller 10 measures time. The time measured by the time keeper 11 is associated with the various detection values (acceleration, angular velocity, and the like) detected by the detector 30. Accordingly, the controller 10 can acquire the various detection values detected by the detector 30 as time series data with which detected times are associated.

The acquirer 12 acquires the exercise data related to the exercise of the user, such as the acceleration, the angular velocity, and the like detected by the detector 30. The time measured by the time keeper 11 is associated with each piece of the exercise data. Accordingly, the exercise data is acquired as time series data. The acquirer 12 functions as acquisition means.

The determiner 13 determines, on the basis of data indicating the acceleration (acceleration in specific direction) along the axis of a specific direction (for example, the gravity direction) included in the exercise data acquired by the acquirer 12, a presence or absence of an object (the pouch 201 or the like) that collides with the detector 30 (determines the presence or absence of noise caused by collision). The manner in which the determiner 13 performs this determination is described later. The determiner 13 functions as determination means. Note that, herein, the phrase “collides with the detector 30” should be construed as including the object colliding with a device (for example, the information processing device 100) that includes the detector 30.

The calculator 14 analyzes the exercise data acquired by the acquirer 12 to calculate the index (for example, the magnitude of vertical movement of the body, the rotation angle of the waist, and the like) related to the exercise of the user. The calculator 14 functions as calculation means. Note that the calculator 14 changes the index calculation method in accordance with the result of the determination of the presence or absence of noise (the presence or absence of the object (the pouch 201 or the like)) carried out by the determiner 13. That is, the calculator 14 calculates the index using a normal calculation method when there is no object that collides with the detector 30 (when there is no noise). However, when there is an object that collides with the detector 30 (when there is noise), the calculator 14 changes to an index calculation method that enables calculation of the index by masking the exercise data influenced by the collision of the object, decreasing the contribution rate to the index calculation, and the like.

When calculating the index, the calculator 14 may collect a large amount of the exercise data, perform deep learning, and use the results of the deep learning to calculate the index. In such a case, the deep learning is carried out by training with large amounts of training data for times when there is no object (the pouch 201 or the like) that collides with the detector 30 (when there is no noise) and training data for times when there is an object (the pouch 201 or the like) that collides with the detector 30 (when there is noise).

Due to the calculator 14 changing the index calculation method depending on the presence or absence of the object (the presence or absence of noise) determined by the determiner 13, the information processing device 100 can calculate, with relatively high accuracy, the index related to the exercise of the user regardless of whether there is or is not an object that collides (interferes) with the detector 30 (regardless of whether there is or is not noise in the exercise data values acquired by the detector 30 (the acceleration sensor 31 or the like)).

Next, the manner in which the determiner 13 determines the presence or absence of the object (the pouch 201 or the like) that collides with the detector 30 (the presence or absence of noise) is described.

As illustrated in FIG. 1, when the user performs exercise such as running or the like while wearing both the information processing device 100 (sensor device) and the pouch 201 (object) on the waist, the pouch 201 collides with the information processing device 100 from behind each time a foot of the user makes ground contact. Moreover, due to this collision, for the direction forward from the front of the user (the Y-axis direction), impact from back to front is applied. As such, when behind the user is a positive value, the absolute value of the negative vale of the Y-axis direction acceleration acquired by the acquirer 12 increases.

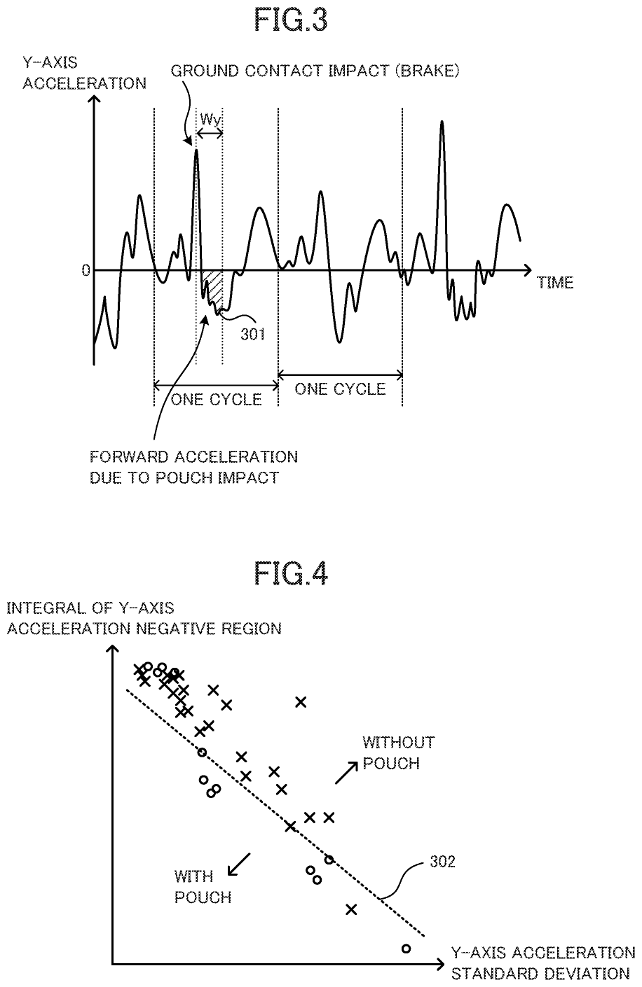

When running, a brake is applied each time a foot of the user makes ground contact, thereby generating backward acceleration in the Y-axis direction, and acceleration occurs each time a foot kicks off, thereby generating forward acceleration in the Y-axis direction. Accordingly, when expressing the Y-axis direction acceleration on a graph along a time series, as illustrated in FIG. 3, a cycle is repeated in which a maximum positive value is obtained at the timing of ground contact and, thereafter, a negative value is obtained due to collision of the pouch 201 or kicking off of a foot. It is thought that the braking at ground contact and the acceleration due to kicking off of a foot in the graph are mostly unaffected by the presence or absence of the pouch 201. Accordingly, it is thought that the area of the negative region of the graph increases due to collision of the pouch 201 only when the pouch 201 is present.

Thus, by analyzing the acceleration, it is possible to calculate the length of one cycle of exercise (for example, when running, the amount of time from when one foot kicks off to when the other foot kicks off).

Here, in order to facilitate understanding of the change in acceleration caused by the impact of ground contact, the timing at which the acceleration reaches a maximum value in one cycle of exercise is regarded as the timing of ground contact, and the area of a negative region 301 (the diagonally hatched portion illustrated in FIG. 3) is calculated for data indicating acceleration in a duration of a first reference time Wy (for example, a constant value of 0.02 seconds to 0.1 seconds) from the timing of ground contact. It is thought that the area of the negative region 301 becomes larger (since the region is a negative region, becomes smaller as an integral value) when the pouch 201 is present. Note that, a constant value such as described above may be used as the first reference time Wy, or a value obtained by dividing the time of one cycle by a first reference number (for example, a constant value of 3 to 8) may be used.

However, since the size that the integral value of the negative region 301 becomes varies depending on the manner of running (running style) of the user, the standard deviation is also used as described below. It is thought that, if the pouch 201 is absent, the Y-axis acceleration values will be relatively stable. However, it is also thought that, if the pouch 201 is present, the Y-axis acceleration will be unstable. Moreover, if the values are unstable, the value of the standard deviation will become larger. That is, as unstable collisions with large standard deviation (for example, collisions by the pouch 201) increase, the area of the negative region becomes larger and, conversely, as monotonous collisions with small standard deviation (for example, impact due to ground contact when the pouch 201 is absent) increase, the area of the negative region becomes smaller.

Accordingly, for the acceleration values in the Y-axis direction, the relationship between the standard deviation and the integral of the negative region can be used, and a determination can be made that the pouch 201 is present (noise is present) when the area of the negative region is relatively large (since the region is a negative region, the integral value is small) with respect to a certain standard deviation.

FIG. 4 illustrates the result of plotting, on a scatter diagram, the standard deviation and the integral of the negative region of the data indicating acceleration obtained in experiments in which the information processing device 100 is worn on the waist and actual Y-axis direction acceleration values are obtained for cases in which the pouch 201 is worn on the waist (with the pouch 201) and for cases in which the pouch 201 is not worn on the waist (without the pouch 201). In FIG. 4, data for cases with the pouch 201 is indicated with an “o” symbol, and data for cases without the pouch 201 is indicated with an “x” symbol. These multiple pieces of data are used as training data to train a classifier (first classifier) such as a support vector machine (SVM), for example and, as a result, the trained classifier can determine the presence or absence of the pouch 201. By using the trained classifier (the first classifier), the determiner 13 can, for example, determine that the data to the upper right of the dashed line 302 illustrated in FIG. 4 is “without the pouch”, and that the data to the lower left of the dashed line 302 is “with the pouch.”

Returning to FIG. 1, it is thought that the pouch 201 collides with the information processing device 100 from behind each time a foot makes ground contact when running or the like, and that the gravity direction (the Z-axis direction) acceleration values acquired by the acquirer 12 become unstable due to this collision. Moreover, when these values become unstable, the standard deviation becomes larger. Additionally, when running, a large acceleration occurs from down to up at the timing at which a foot makes ground contact, the effects of gravitational acceleration are canceled out while the body prior to ground contact is naturally falling, and the acceleration is a value close to about 0.

Accordingly, when expressing, on a graph, of the Z-axis direction acceleration along a time series with vertically upward as a positive value, as illustrated in FIG. 5, impact from down to up is received at the timing of ground contact, and a maximum positive value is obtained as the acceleration. Thus, a graph is obtained in which, a cycle is repeated at each ground contact in which unstable small values caused by collisions of the pouch 201 (for example, the pouch 201 collides with the information processing device 100 from above) are obtained before and after the ground contact.

For the Z-axis as well, in order to facilitate understanding of the change in acceleration caused by the impact of ground contact, the timing at which the maximum value is reached in one cycle of exercise is regarded as the timing of ground contact, and the standard deviation is calculated for the data indicating acceleration in a duration of a second reference time ±Wz (for example, a constant value of 0.05 seconds to 0.2 seconds) from the timing of ground contact. It is thought that, since the acceleration values are unstable when the pouch 201 is present, the standard deviation becomes larger. Note that, a constant value such as described above may be used as the second reference time Wz, or a value obtained by dividing the time of one cycle by a second reference number (for example, a constant value of 2 to 7) may be used.

However, since the size that the standard deviation becomes varies depending on the running style of the user, the median is also used as described below. For the Z-axis direction acceleration, since impact from up to down is often received (becomes acceleration with a negative value) when the pouch 201 collides with the information processing device 100, many small values caused by impacts other than ground contact impacts become included in the ±Wz duration and the median becomes smaller. In contrast, when there are no impacts of the pouch 201, the proportion of ground contact impacts in the ±Wz duration increases (there are many instances where maximum acceleration is reached in one cycle), and the median becomes larger.

Accordingly, for the Z-axis direction acceleration values, the relationship between the standard deviation and the median can be used, and a determination can be made that the pouch 201 is present when the standard deviation is relatively large with respect to a certain median.

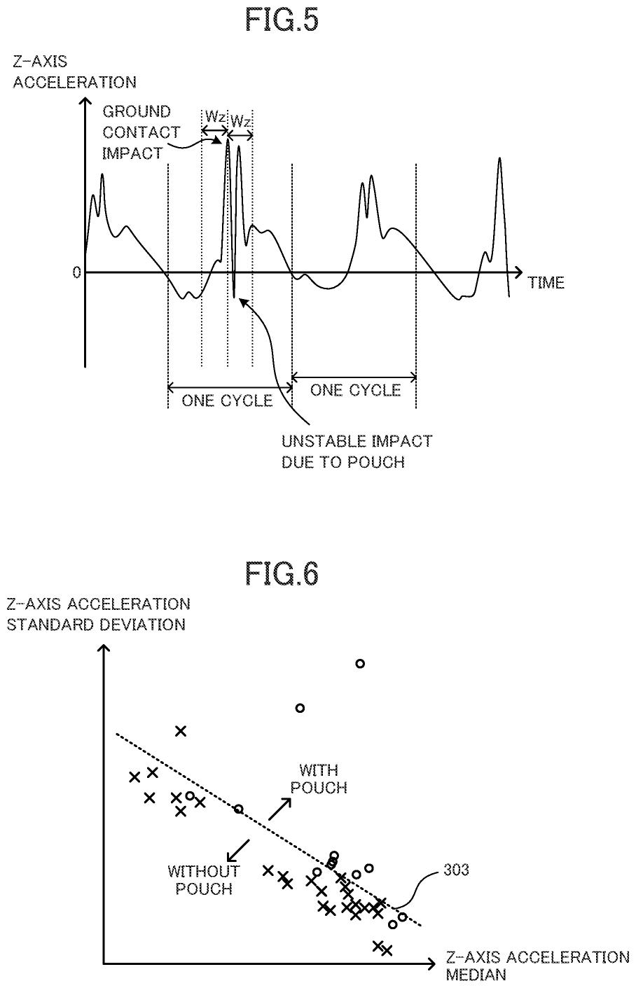

FIG. 6 illustrates the result of plotting, on a scatter diagram, the standard deviation and the median of data indicating acceleration obtained in experiments in which the information processing device 100 is worn on the waist and actual Z-axis direction acceleration values are obtained for cases in which the pouch 201 is worn on the waist (with the pouch 201) and for cases in which the pouch 201 is not worn on the waist (without the pouch 201). In FIG. 6, data for cases with the pouch 201 is indicated with an “o” symbol, and data for cases without the pouch 201 is indicated with an “x” symbol. These multiple pieces of data are used as training data to train a classifier (second classifier) such as a support vector machine, for example, and the trained classifier can determine the presence or absence of the pouch 201. By using the trained classifier (the second classifier), the determiner 13 can, for example, determine the data to the upper right of the dashed line 303 illustrated in FIG. 6 is “with the pouch”, and the data to the lower left of the dashed line 303 is “without the pouch.”

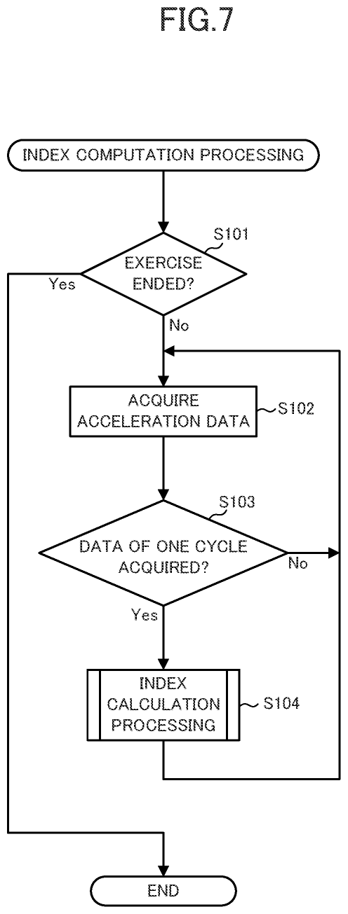

Next, index computation processing by the controller 10 is described while referencing FIG. 7. The information processing device 100 starts the index computation processing when instructed by the user to start the index computation processing.

Firstly, the controller 10 determines whether the exercise of the user is ended (step S101). For example, the controller 10 determines that the exercise is ended if, for a predetermined amount of time (for example, three seconds), the detector 30 detects only small acceleration and angular velocity values that are thought to indicate that the user is not exercising. The controller 10 determines that the exercise is ended also when the user instructs stopping of the index computation processing via the UI 50.

If the controller 10 determines that the exercise of the user is ended (step S101; Yes), the controller 10 ends the index computation processing. If the controller 10 determines that the exercise of the user is not ended (step S101; No), the acquirer 12 acquires the data indicating acceleration detected by the acceleration sensor 31, and accumulates the acquired data together with the time measured by the time keeper 11 (the timing at which that data is detected) in the storage 20 (step S102). Step S102 is also called an “acquisition step.”

Then, the calculator 14 analyzes the data accumulated to that point in the storage 20 to determine whether data of one cycle of exercise has been acquired (step S103). If the data of one cycle has not been acquired (step S103; No), step S102 is executed. If the data of one cycle has been acquired (step S103; Yes), indicator calculation processing (described later) is performed (step S104) and, then, step S102 is executed.

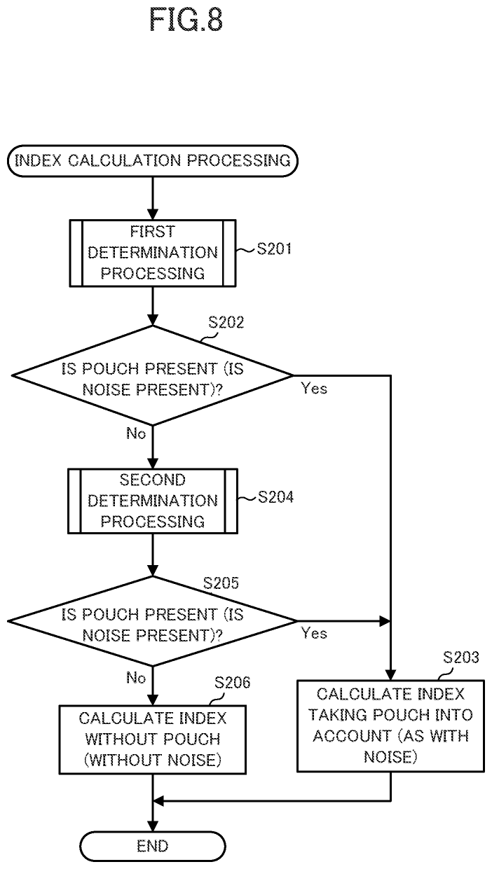

Next, the indicator calculation processing executed in step S104 is described while referencing FIG. 8. Firstly, the determiner 13 performs a first determination processing (described later) (step S201). In the first determination processing, the determiner 13 determines if the pouch 201 (object) is present or absent (if noise is present or absent) (step S202).

If a determination is made that the pouch 201 is present (noise is present) (step S202; Yes), the calculator 14 calculates the index related to the exercise of the user taking into account the wearing of the pouch 201 (that noise is present) (step S203), and ends the indicator calculation processing. If a determination is made that the pouch 201 is absent (noise is absent) (step S202; No), the determiner 13 performs a second determination processing (described later) (step S204). In the second determination processing, the determiner 13 determines if the pouch 201 (object) is present or absent (if noise is present or absent) (step S205).

If a determination is made that the pouch 201 is present (noise is present) (step S205; Yes), step S203 is executed. If a determination is made that the pouch 201 is absent (noise is absent) (step S205; No), the calculator 14 calculates the index related to the exercise of the user without taking into account the wearing of the pouch 201 (noise is absent) (step S206), and ends the indicator calculation processing. Note that, steps S201 and S204 are also called “determination steps.”

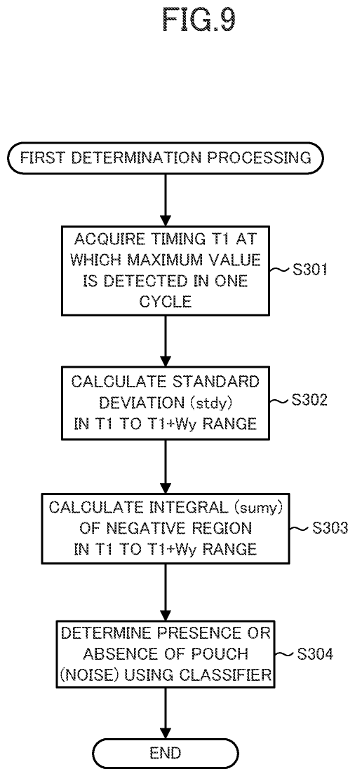

Next, the first determination processing executed in step S201 is described while referencing FIG. 9. Firstly, the determiner 13 references the data indicating Y-axis acceleration that is accumulated in the storage 20, and acquires, as the ground contact timing, a timing T1 at which the maximum value, in the most recent one cycle in the data, is detected (step S301).

Then, from among the data, the determiner 13 calculates a standard deviation stdy of a set (first data group) of data near the ground contact (a first duration from the ground contact timing T1 to the ground contact timing T1+the first reference time Wy) (step S302). Next, the determiner 13 calculates, from among the data, an integral sumy of the negative region (region in the direction forward from the front of the user) near the ground contact (the first duration from the ground contact timing T1) (step S303). In one example, the integral sumy is calculated by integrating the portion corresponding to the negative region 301 illustrated in FIG. 3.

Then, the determiner 13 determines the presence or absence of the pouch 201 (object) (presence or absence of noise) using a pre-trained support vector machine or similar classifier with the standard deviation stdy and the integral sumy as feature quantities (step S304), and ends the first determination processing.

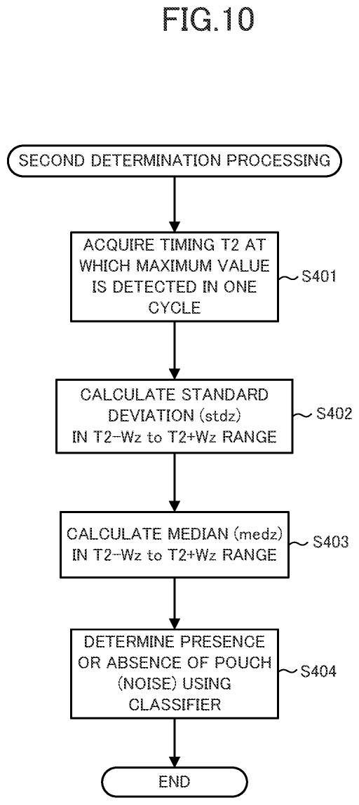

Next, the second determination processing executed in step S204 is described while referencing FIG. 10. Firstly, the determiner 13 references the data indicating Z-axis acceleration that is accumulated in the storage 20, and acquires, as the ground contact timing, a timing T2 at which the maximum value, in the most recent one cycle in the data, is detected (step S401).

Then, from among the data, the determiner 13 calculates a standard deviation stdz of a set (second data group) of data near the ground contact (a second duration from before to after a second reference timing Wz of the ground contact timing T2) (step S402). Next, the determiner 13 calculates, from among the data, a median medz of the set of data near the ground contact (the second duration centered on the ground contact timing T2) (step S403).

Then, the determiner 13 determines the presence or absence of the pouch 201 (object) (presence or absence of noise) using a pre-trained support vector machine or similar classifier with the standard deviation stdz and the median medz as feature quantities (step S404), and ends the second determination processing.

Note that in step S203 of the index calculation processing, an output device or the like of the UI 50 may notify the user that the pouch 201 (noise) is present. Due to this notification, the user can be made aware that the pouch 201 is colliding with the information processing device 100, and can take action such as changing the position where the pouch 201 is worn, removing the pouch 201 and then performing the exercise such as running again, or the like.

Japanese Unexamined Patent Application Publication No. 2016-34479, for example, describes a device attached to the waist of a user. The device detects, from an acceleration value and with high accuracy, the timings and the like at which a user that is running leaves and contacts the ground. Typically, this type of sensor device is worn at a position of the waist of the user. However, the user may wrap the pouch around the waist in order to carry small objects and the like, and run. While it is preferable that the sensor device and the pouch are worn at different (for example left and right) positions of the waist so as not to interfere with each other, in many cases, both the sensor device and the pouch are actually worn at a back position on the waist when running so that the sensor device and the pouch do not get in the way. In such cases, the pouch collides with the sensor device while running, and, due to the influence of these collisions, a phenomenon of some of the running index values detected by the sensor device becoming unintended values has been observed. It is also thought that the collisions against the sensor device are a portion of the noise that obstructs the accurate calculation of the running index values.

However, due to the index computation processing described above, the information processing device 100 of the present embodiment can determine the presence or absence of an object (the pouch 201, or the like) (the presence or absence of noise included in the exercise data acquired by the detector 30 (the acceleration sensor 31, or the like)) that interferes with the detector 30. Therefore, the information processing device 100 can calculate the index while taking into account the presence or absence of the object (the presence or absence of noise).

Note that, in the embodiment described above, running is envisioned as the exercise performed by the user. However, the type of exercise is not limited to running. For example, the present embodiment can be applied to any exercise in which an action of the feet making ground contact is performed cyclically. Examples thereof include walking, jumping rope, and the like.

Additionally, a case is envisioned in which the information processing device 100 is worn on the waist of the user. However, the wearing location is not limited to the waist, and the information processing device 100 may be worn on the neck, a wrist, an ankle, or the like of the user. Even when worn at a location of the body of the user other than the waist, the information processing device 100 can acquire the data related to the exercise of the user. Additionally, even when worn at a location of the body of the user other than the waist, the information processing device 100 can determine, on the basis of the data indicating acceleration included in the exercise data acquired by the acquirer 12, the presence or absence of an object that collides with the detector 30 (the presence or absence of noise included in the exercise data acquired by the detector 30 (the acceleration sensor 31, or the like)).

Additionally, a case is envisioned in which the user wears the pouch 201 with the pouch belt 202 wrapped around the waist. However, the wearing location of the pouch 201 is not limited to the waist, and the pouch 201 may be worn with the pouch belt 202 hung diagonally from the shoulder of the user. In such a case as well, the pouch 201 can collide with the information processing device 100 due to the exercise of the user, and the information processing device 100 can determine, on the basis of the data indicating acceleration included in the exercise data acquired by the acquirer 12, the presence or absence of an object that collides with the detector 30 (the presence or absence of noise included in the exercise data acquired by the detector 30 (the acceleration sensor 31, or the like)).

### Modified Examples

Embodiments of the present disclosure are described above, but these embodiments are merely examples and do not limit the scope of application of the present disclosure. That is, various applications of the embodiments of the present disclosure are possible, and all embodiments are included in the scope of the present disclosure.

For example, in the index calculation processing (FIG. 8), the first determination processing and the second determination processing are performed, but a configuration is possible in which the determination of the presence or absence of the pouch 201 (the object) is made using only one of these two determination processings. For example, when determining the presence or absence of the pouch 201 (the presence or absence of noise) using only the first determination processing, there is no need to perform the second determination processing. Conversely, when determining the presence or absence of the pouch 201 (the presence or absence of noise) using only the second determination processing, there is no need to perform the first determination processing.

A configuration is possible in which the pouch 201 is assumed to be present (noise is assumed to be present) and the index calculation is performed taking the pouch 201 (noise) into account only in cases in which a determination of “pouch is present (noise is present)” is made by both of the two determination processings. That is, a configuration is possible in which index calculation that does not take the pouch 201 (noise) into account is performed when a determination of “pouch is absent (noise is absent)” is made by one of the two determination processings. Additionally, a configuration is possible in which the index calculation is performed on the basis of a value obtained by weight averaging the results of the two determination processings.

In the first determination processing (FIG. 9), the timing T1 at which the maximum value of the data indicating Y-axis acceleration is detected is used as the ground contact timing and, in the second determination processing (FIG. 10), the timing T2 at which the maximum value of the data indicating Z-axis acceleration is detected is used as the ground contact timing. However, the acquisition method of the ground contact timing is not limited thereto. A configuration is possible in which, in the first determination processing and also in the second determination processing, the ground contact timing is acquired on the basis of desired acceleration data and angular velocity data that can be acquired by the detector 30.

In the first determination processing, the value obtained by integrating the negative region (region in the direction forward from the front of the user) of the data indicating Y-axis acceleration in the range from the ground contact timing T1 to T1+Wy, but a configuration is possible in which, as in the second determination processing, the median (the median in the range from the ground contact timing T1 to T1+Wy of the data indicating Y-axis acceleration) is used as the feature quantity. In contrast, a configuration is possible in which, in the second determination processing, a value obtained by integrating the negative region is used instead of the median.

Additionally, the range of the data from which the feature quantity is obtained is set to from the ground contact timing T1 to T1+Wy for the Y-axis acceleration, and is set to from the ground contact timing T2−Wz to T2+Wz for the Z-axis acceleration, but the ranges are not limited thereto. For example, a configuration is possible in which the range is set to from the ground contact timing T1−Wy to T1+Wy for the Y-axis acceleration, and is set to from the ground contact timing T2 to T2+Wz for the Z-axis acceleration.

In the first determination processing (FIG. 9) and the second determination processing (FIG. 10), when making the determination of the presence or absence of the pouch 201 (the presence or absence of noise), each of the standard deviation of the data indicating Y-axis acceleration and the standard deviation of the data indicating Z-axis acceleration is used. However, this is merely an example, and the present disclosure is not limited thereto. For example, a configuration is possible in which a distribution of the data indicating Y-axis acceleration and a distribution of the data indicating Z-axis acceleration are used as values indicating variation of the data.

Additionally, configurations are possible in which the type of exercise (running, walking, jumping rope, or the like), the nature of the ground (concrete, dirt, flooring, rubber mat, or the like), the gradient (flat, uphill, downhill, or the like), user habits (ground contact method, running speed, or the like), and the like are analyzed, and the more accurate of the determination based on the Y-axis acceleration and the determination based on the Z-axis acceleration is used, or a weighted sum of values obtained by quantifying both determination results is used (for example, the distance of a boundary line from the support vector machine in a scatter diagram such as illustrated in FIG. 4 and FIG. 6 is used) to make the determination.

Additionally, a configuration is possible in which the information processing device 100 does not include the detector 30, and the information processing device 100 acquires, by wireless communication or the like and in real time or after the exercise from a separate device (sensor device) including the detector 30, a detection value (sensor data) detected by the detector 30 to perform the index computation processing. In such a case, provided that the sensor device provided with the detector 30 is worn on the waist or the like of the user, the location holding the information processing device 100 that that is not provided with the detector 30 is not limited.

Additionally, a description is given in which the information processing device 100 includes the communicator 40 that is a communication interface that outputs data such as the calculated index and the like to an external device. However, the present disclosure is not limited thereto. A configuration is possible in which the information processing device 100 includes an interface from which a non-transitory recording medium can be detached, the data is stored in the non-transitory recording medium, and the non-transitory recording medium is connected to an external device to provide the data such as the index and the like to the external device.

Additionally, in the embodiment of the present disclosure, as an example of the processing method for determining the presence or absence of noise caused by collisions, a processing method is described for a situation in which the pouch 201 collides with the information processing device 100 when the pouch 201 is worn on the waist by the pouch belt 202. However, the present disclosure is not limited thereto. For example, the present disclosure can be applied to a case in which a backpack carried by the user, clothing worn by the user, a part of the body of a person in the surroundings of the user wearing the information processing device 100, an object that comes flying from the surroundings of the user wearing the information processing device 100, or the like collides with the information processing device 100. Additionally, the present disclosure can be applied to a case in which the information processing device 100 worn on the waist of the user collides with a part (for example, the back, waist, hand, or the like) of the body of the user themself. Moreover, even if there is no collision with the information processing device 100 itself, there is a possibility that vibrations from when the pouch collides, due to the impact of ground contact of the user, with a part (the back, waist, or the like) of the body of the user will propagate to the information processing device 100. However, the present disclosure can be applied in this case as well.

Note that, the various functions of the information processing device 100 can be implemented by a computer such as a typical personal computer (PC). Specifically, in the embodiments described above, examples are described in which the programs, such as the index computation processing, performed by the information processing device 100 are stored in advance in the ROM of the storage unit 20. However, a computer may be configured that is capable of realizing these various features by storing and distributing the programs on a non-transitory computer-readable recording medium such as a compact disc read-only memory (CD-ROM), a digital versatile disc (DVD), a magneto-optical disc (MO), a memory card, and universal serial bus (USB) memory, and reading out and installing these programs on the computer.

Furthermore, the program can be superimposed on a carrier wave and applied via a communication medium such as the internet. For example, the program may be posted to and distributed via a bulletin board system (BBS) on a communication network. Moreover, a configuration is possible in which the processing described above is executed by starting the programs and, under the control of the operating system (OS), executing the programs in the same manner as other applications/programs.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. An information processing device, comprising:
at least one processor configured to
acquire exercise data related to exercise of a user from a sensor that detects the exercise data, and
determine, based on data that is included in the acquired exercise data and that indicates acceleration in a specific direction, a presence or absence of noise caused by a collision.

2. The information processing device according to claim 1, wherein the presence or absence of noise caused by a collision includes a presence or absence of noise caused by an object colliding with the user or the sensor, or noise caused by the sensor colliding with a part of a body of the user.

3. The information processing device according to claim 1, wherein the at least one processor is configured to determine the presence or absence of the noise based on at least one of data indicating acceleration in a direction forward from a front of the user and data indicating acceleration in a gravity direction included in the exercise data.

4. The information processing device according to claim 3, wherein the at least one processor is configured to determine the presence or absence of the noise based on at least one of the data indicating the acceleration on an axis in the direction forward from the front of the user near a ground contact timing, that is a timing of the exercise of the user at which ground contact is made, and the data indicating the acceleration on an axis in the gravity direction near the ground contact timing included in the exercise data.

5. The information processing device according to claim 4, wherein
the at least one processor is configured to determine the presence or absence of the noise by at least one of a first classifier and a second classifier,
the at least one processor extracting, as a first data group, the data detected in a range of a first duration that includes the ground contact timing as the data indicating the acceleration on the axis in the direction forward from the front of the user near the ground contact timing,
the first classifier being trained in advance with, as feature quantities, an index indicating a variation of the data of the first data group and a value obtained by integrating, in the first data group, a region in the direction forward from the front with a range of the first duration,
the at least one processor extracting, as a second data group, the data detected in a range of a second duration that includes the ground contact timing as the data indicating the acceleration on the axis in the gravity direction near the ground contact timing,
the second classifier being trained in advance with, as feature quantities, an index indicating a variation of the data of the second data group and a median.

6. The information processing device according to claim 5, wherein the classifier is a support vector machine based classifier.

7. The information processing device according to claim 1, wherein a device provided with the sensor is worn on a waist of the user.

8. The information processing device according to claim 1, wherein
the at least one processor is configured to
calculate an index related to the exercise of the user from the acquired exercise data, and
change a calculation method of the index in accordance with a result of the determination of the presence or absence of the noise.

9. The information processing device according to claim 1, further comprising:
an output device that, when, as a result of the determination of the presence or absence of the noise, a determination is made that noise is present, notifies that the noise is present.

10. An information processing method of an information processing device, the method comprising:
acquiring exercise data related to exercise of a user from a sensor that detects the exercise data; and
determining, based on data that is included in the acquired exercise data and that indicates acceleration in a specific direction, a presence or absence of noise caused by a collision.

11. A non-transitory recording medium that storing a program that causes at least one processor of an information processing device to execute:
acquiring exercise data related to the exercise of a user from a sensor that detects the exercise data; and
determining, based on data that is included in the acquired exercise data and that indicates acceleration in a specific direction, a presence or absence of noise caused by a collision.
